(19) United States
(12) Patent Application Publication
(10) Pub. No.: US 2009/0238716 A1
(43) Pub. Date: Sep. 24, 2009

(54) AIRBORNE PATHOGEN DISINFECTANT SYSTEM AND METHOD

(76) Inventor: Richard W. Weening, West Palm Beach, FL (US)

Correspondence Address: GODFREY & KAHN S.C., 780 NORTH WATER STREET, MILWAUKEE, WI, 53202, US

(21) Appl. No.: 12/359,283

(22) Filed: Jan. 24, 2009

Related U.S. Application Data
(63) Continuation of application No. 61/039,039, filed on Mar. 24, 2008.

(51) Int. Cl. A61L 2/18; A61L 2/24

(52) U.S. Cl. 422/3; 422/28; 422/292; 422/105

(57) ABSTRACT

A method and device of treating an enclosed space with a liquid disinfecting agent. A device diffuses small droplets of the liquid into the space and the droplets evaporate to leave gaseous disinfecting agent within the space to be treated. The concentration of the agent within the space may be preferably maintained at or near a saturation concentration for maximum efficiency and effectiveness. Operation of the device may be varied to match the atmospheric conditions within the space and the current level of concentration of the agent within the space.

### CROSS-REFERENCE TO RELATED APPLICATION

The present application claims priority to U.S. Provisional Application Ser. No. 61/039,039, filed Mar. 24, 2008, the disclosure of which is incorporated herein by reference.

### BACKGROUND

In the early 1940's it was discovered that triethylene glycol (“TEG”) vapor had a lethal effect on air-borne bacteria and viruses. In 1943 several researchers reported¹ results of an investigation to confirm the antimicrobial action of glycols for air-borne disease agents. In this investigation, TEG was selected as the most lethal antimicrobial and the least toxic humans. Two experiments were conducted on the efficacy of TEG. The first tested the biocidal efficacy of TEG vapor on Beta Hemolytic Streptococcus Group A and Pneumonococcus Type I, The second tested the viricidal efficacy using TEG vapor in chambers with influenza virus and comparing the results to chambers without TEG. All the mice lived in the TEG treated chambers. All the mice died in the untreated chamber. ¹ O. H. Robertson, Theodore T. Puck, Henry F. Lemon, Clayton G. Loosli; Science Magazine (Vol., 97, No. 2510, Feb. 5, 1943)

It is believed that this promising research did not progress to commercial use for several reasons. First, at the time there was little information on the toxicity of TEG though some preliminary tests suggested it was the least toxic of the glycols.² Comprehensive toxicity tests would not be conducted for several decades. Second, while TEG can be vaporized in an enclosed laboratory environment, there was no easy way to continuously convert TEG into a vapor in precise and controllable quantities necessary for continuous disinfection of a space. TEG is very non volatile with a vapor pressure of less than 0.01 (mm HG) at 68° F. and a boiling point at 575° F. By comparison, the vapor pressure of ethanol at the same temperature is 48.5 and it boils 173° F.³ The low vapor pressure and high temperatures needed to create TEG vapor caused early researchers to soak bed sheets with TEG, cure them overnight and hang them in test chambers to serve as a source of vapor. Third, and closely related, is the issue of controlling the quantity of TEG relative to the size of the area being treated. TEG is effective as an antimicrobial at known concentration levels in the air. In the confirmation investigations referenced above the size of the area to be treated was known and a specific quantity of liquid TEG matched to the cubic area of the test room was heated and vaporized to sterilize the area as a one-time event. Using conventional approaches, sterilizing an area not one-time but continuously (as would be required in commercial application) would require continuous injection of controlled quantities of TEG vapor to maintain the required concentration level. ² W. M. Lauter and V. L. Vrla, Journal America Pharmaceutical Association, Vol. 29: No. 5, 1940,³ A liquid at any temperature exerts a pressure on its environment. This pressure, the vapor pressure, results from molecules leaving the surface of the liquid to become vapor and occurs because the molecules are in constant motion. This pressure, the vapor pressure, results from molecules leaving the surface of the liquid to become vapor and occurs because the molecules are in constant motion. (molecules)liquid>>(molecules)vapor. As a liquid is heated, its kinetic energy increases; the equilibrium shifts to the right and more molecules move into the gaseous state, thereby increasing the vapor pressure. The boiling point of a pure liquid is therefore defined as the temperature at which the vapor pressure of the liquid exactly equals the pressure exerted on it by the atmosphere.

In summary, the absence of definitive toxicity information as well as methods for producing TEG vapor in controlled quantities we believe to have prevented the development of TEG as a practical method of sterilizing the air of interior spaces. Improvements to the ability to distribute TEG within a space and to precisely control the amount of TEG released within the space are desirable.

### BRIEF DESCRIPTION OF THE DRAWINGS

The accompanying drawings, which are incorporated in and constitute a part of the description, illustrate several aspects of the invention and together with the description, serve to explain the principles of the invention. A brief description of the drawings is as follows:

FIG. 1 is a perspective view of a liquid diffusion device according to the present disclosure.

FIG. 2 is a top view of the liquid diffusion device of FIG. 1.

FIG. 3 is a perspective view of the liquid diffusion device of FIG. 1 with a cover exploded.

FIG. 4 is a perspective view of the liquid diffusion device of FIG. 1 with the cover removed.

FIG. 5 is a perspective view of a liquid cartridge for use with the liquid diffusion device of FIG. 1.

FIG. 6 is a top view of the cartridge of FIG. 5.

FIG. 7 is a bottom view of the cartridge of FIG. 5.

FIG. 8 is a first exploded perspective view of the cartridge of FIG. 5, with a tube and venturi assembly removed for clarity.

FIG. 9 is a second exploded perspective view of the cartridge of FIG. 8.

FIG. 10 is an exploded side view of the cartridge of FIG. 8.

FIG. 11 is a top perspective view of a top cap for the cartridge of FIG. 8.

FIG. 12 is a bottom perspective view of the top cap of FIG. 11.

FIG. 13 is a top view of the top cap of FIG. 1.

FIG. 14 is a bottom view of the top cap of FIG. 11.

FIG. 15 is a perspective view of a baffle of the cartridge of FIG. 8.

FIG. 16 is a top view of the baffle of FIG. 15.

FIG. 17 is a bottom view of the baffle of FIG. 15.

FIG. 18 is a first side view of the baffle of FIG. 15.

FIG. 19 is a second side view of the baffle of FIG. 15.

FIG. 20 is a side cross-section view of the baffle of FIG. 15, taken along line 20-20 of FIG. 16.

FIG. 21 is a perspective view of a reservoir of the cartridge of FIG. 8.

FIG. 22 is a top perspective view of the reservoir of FIG. 21.

FIG. 25 is a side perspective view of a tube and venturi assembly of the cartridge of FIG. 8.

FIG. 26 is a side view of the tube and venturi assembly of FIG. 25.

FIG. 27 is an exploded side view of the tube and venturi assembly of the cartridge of FIG. 25.

FIG. 28 is a side cross-sectional view of the tube and venturi assembly of the cartridge of FIG. 25.

FIG. 29 is a front cross-sectional view of the tube and venturi assembly of FIG. 25.

FIG. 30 is a bottom view of the tube and venturi assembly of FIG. 25.

FIG. 31 is a perspective view of the nozzle cap of venturi assembly of FIG. 25.

FIG. 32 is a first side view of the nozzle cap of FIG. 31.

FIG. 33 is a second side view of the nozzle cap of FIG. 31.

FIG. 34 is a cross-sectional view of the cartridge of FIG. 5, illustrating the flow of gas into the cartridge, through the venturi and into the headspace.

FIG. 35 is a cross-sectional view of the cartridge of FIG. 5, illustrating the flow of gas and diffused liquid from the headspace through the baffle and out of the cartridge.

FIG. 36 is a cross-sectional view of the cartridge of FIG. 5, illustrating the connection between the baffle, the cap and the reservoir.

FIG. 37 is a perspective cross-sectional view of an alternative embodiment of a liquid diffusion device according to the present disclosure with an anti-spill feature.

FIG. 38 is a perspective view of a further embodiment of a replaceable liquid cartridge according to the present disclosure.

FIG. 39 is a side view of the cartridge of FIG. 38.

FIG. 40 is a rear view of the cartridge of FIG. 38.

FIG. 41 is a top view of the cartridge of FIG. 38.

FIG. 42 is an exploded perspective view of a cartridge head assembly for the cartridge of FIG. 38.

FIG. 43 is a perspective view of an alternative embodiment of a diffusion device according to the present disclosure.

FIG. 44 is a perspective view of the diffusion device of FIG. 43, with the cover removed and resting adjacent the base.

### DETAILED DESCRIPTION

Reference will now be made in detail to exemplary aspects of the present invention which are illustrated in the accompanying drawings. Wherever possible, the same reference numbers will be used throughout the drawings to refer to the same or like parts.

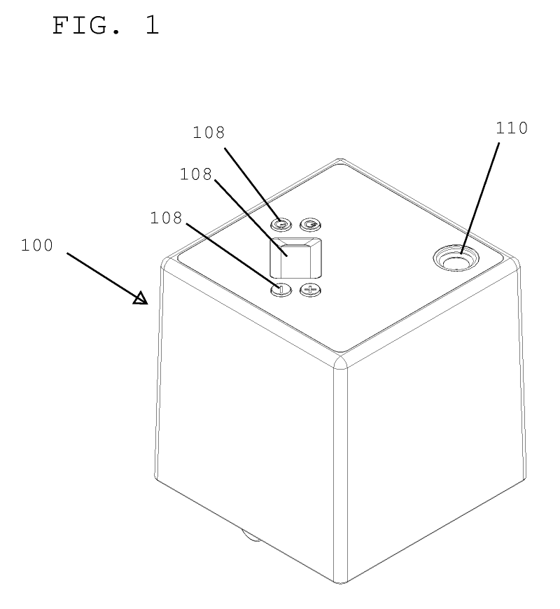

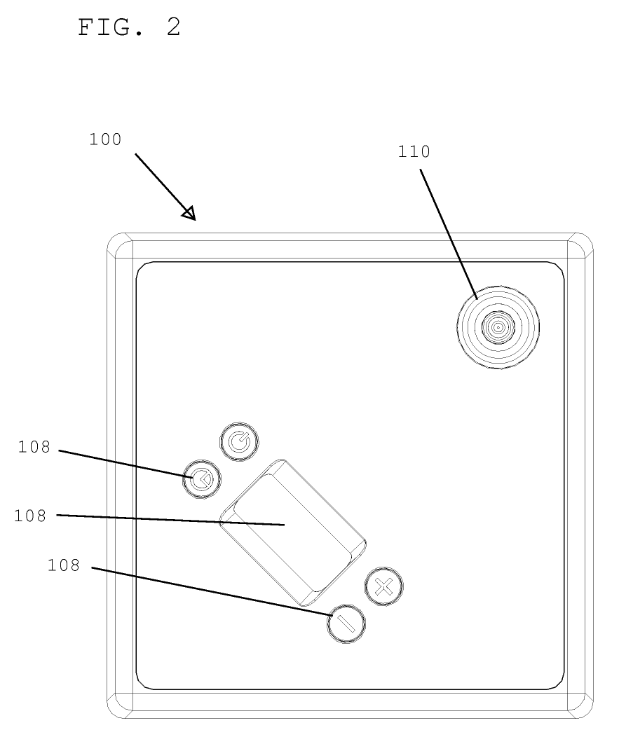

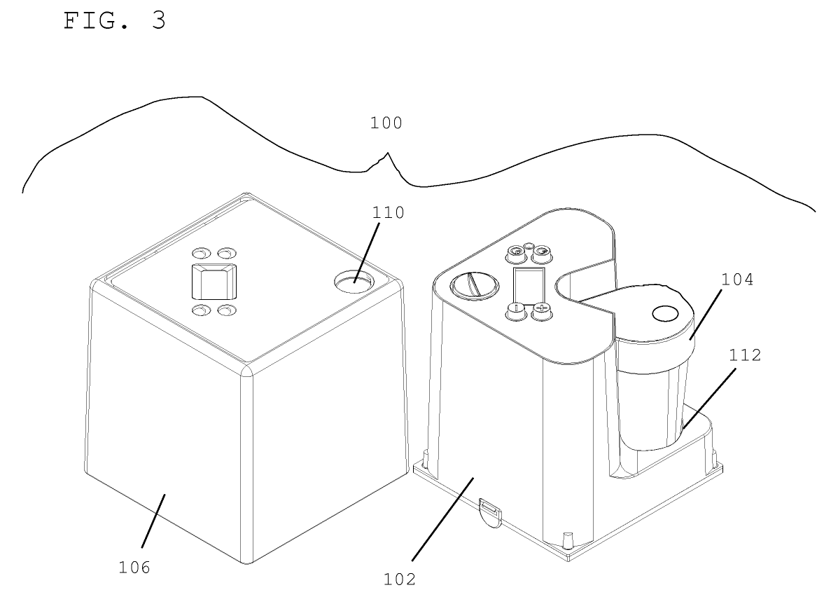

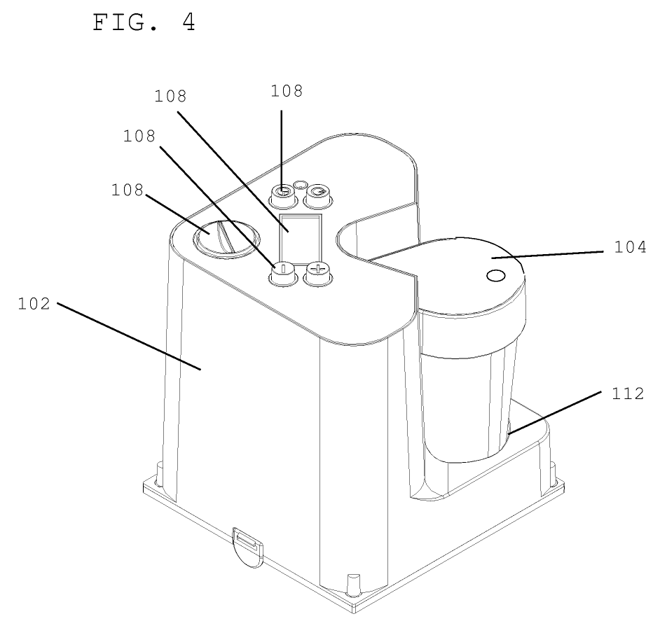

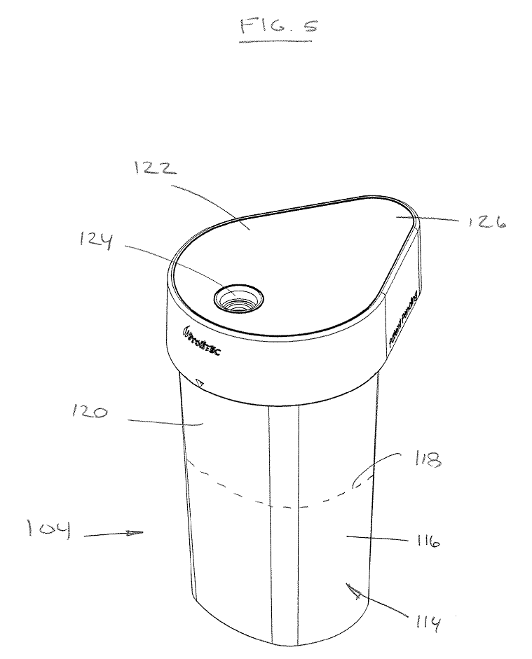

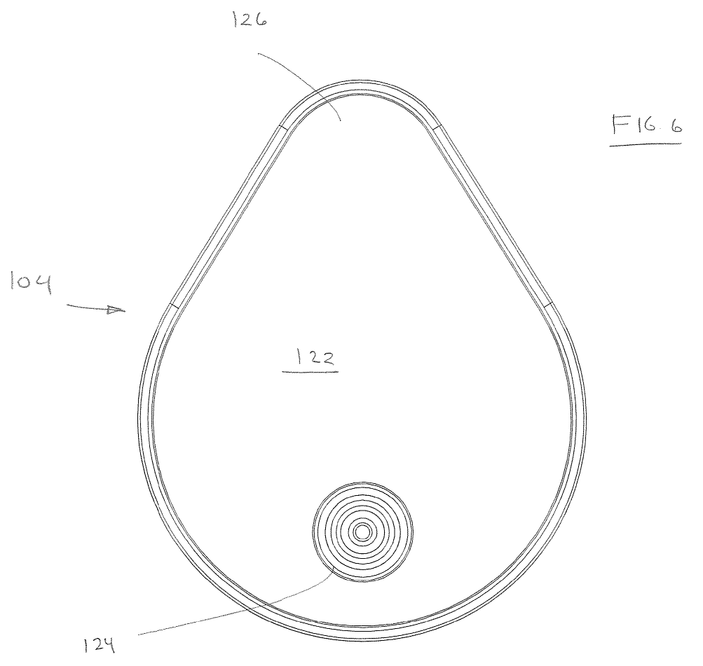

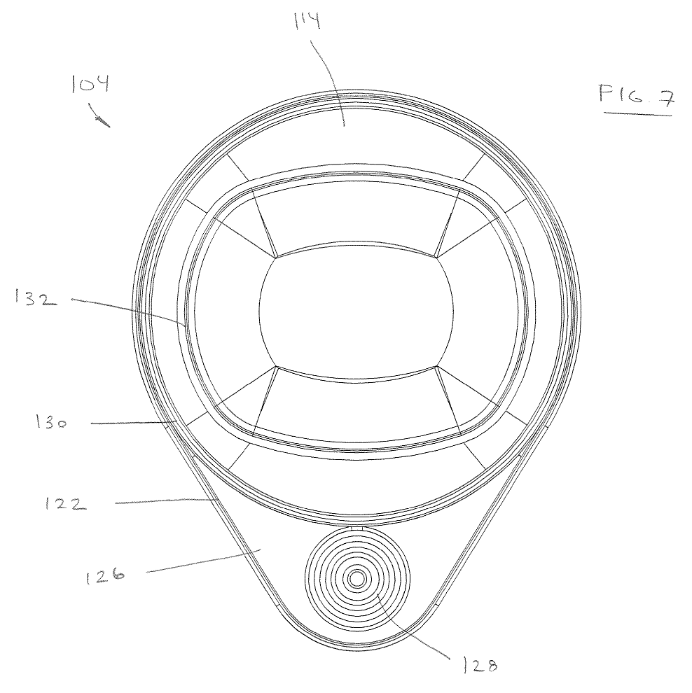

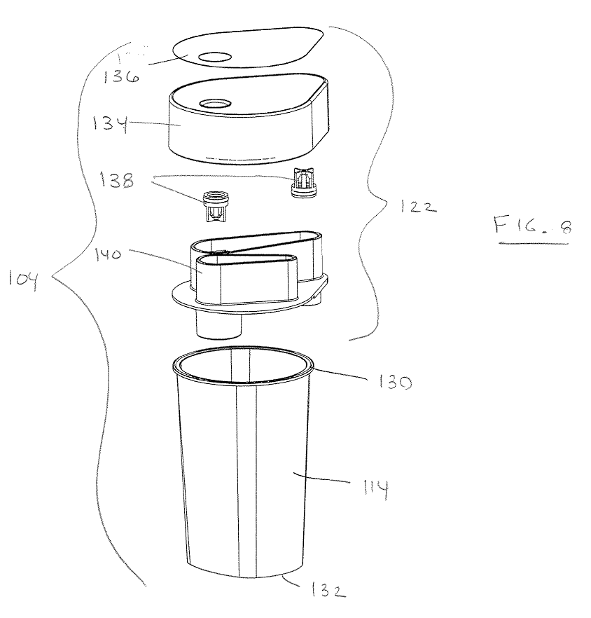

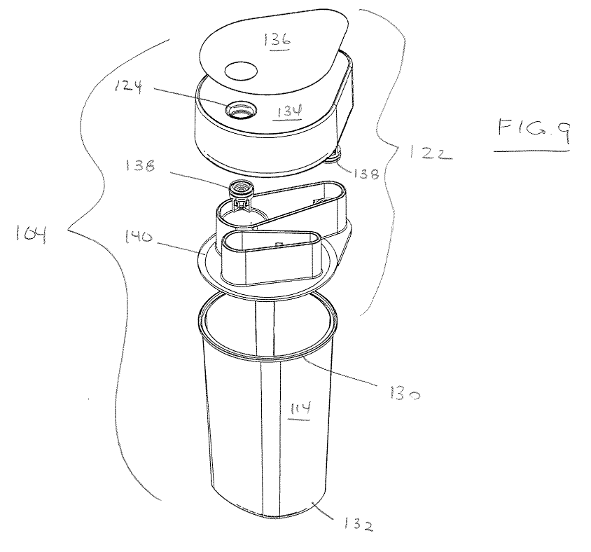

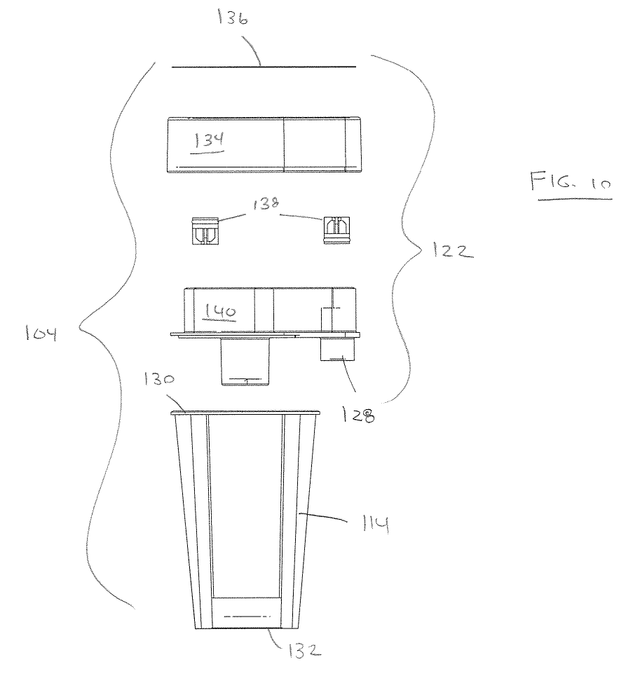

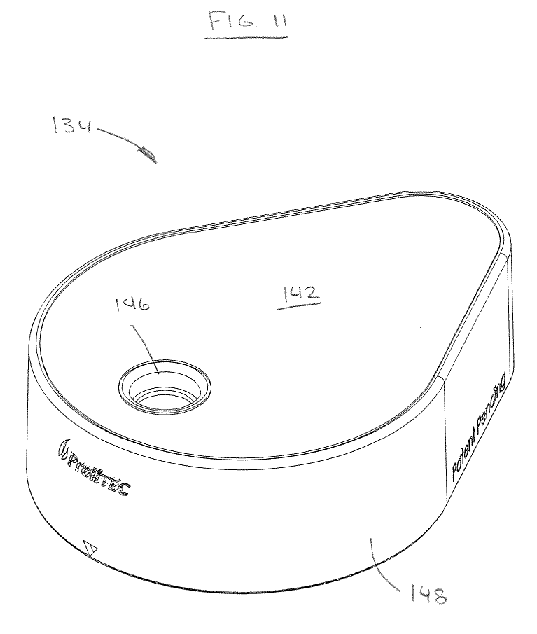

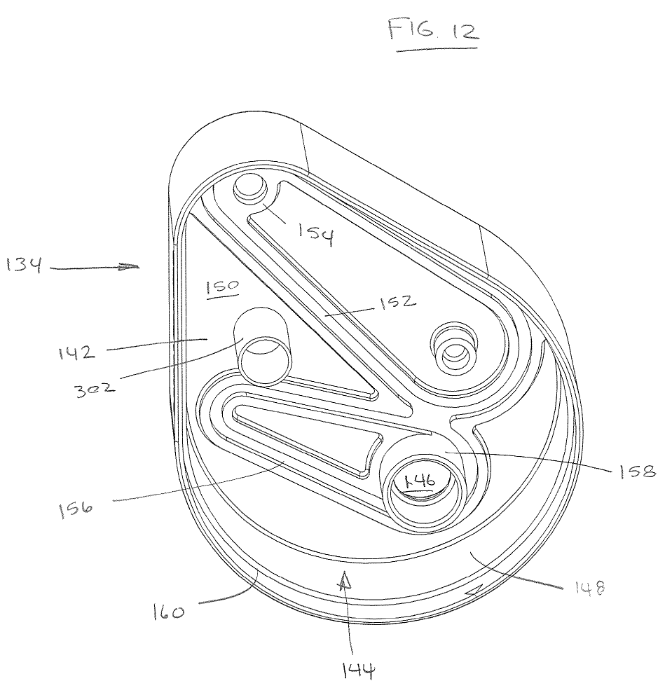

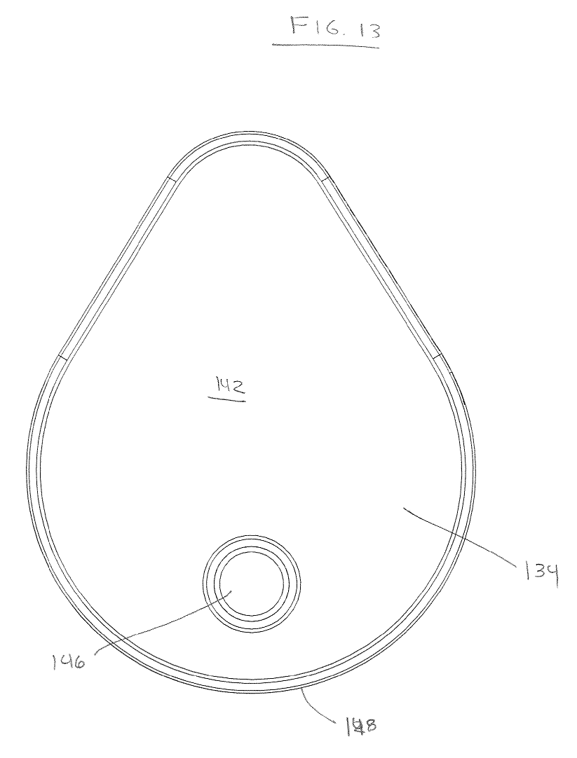

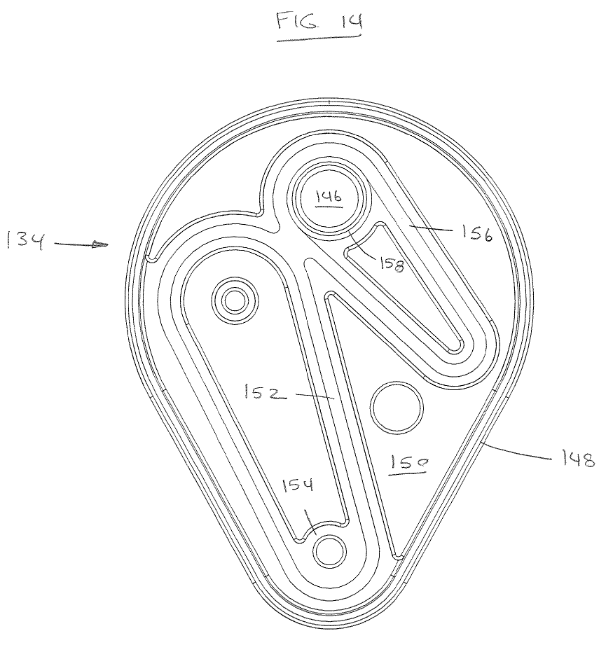

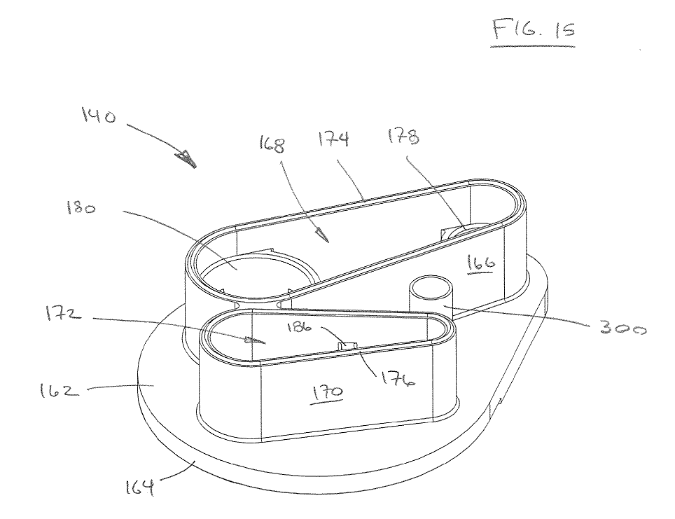

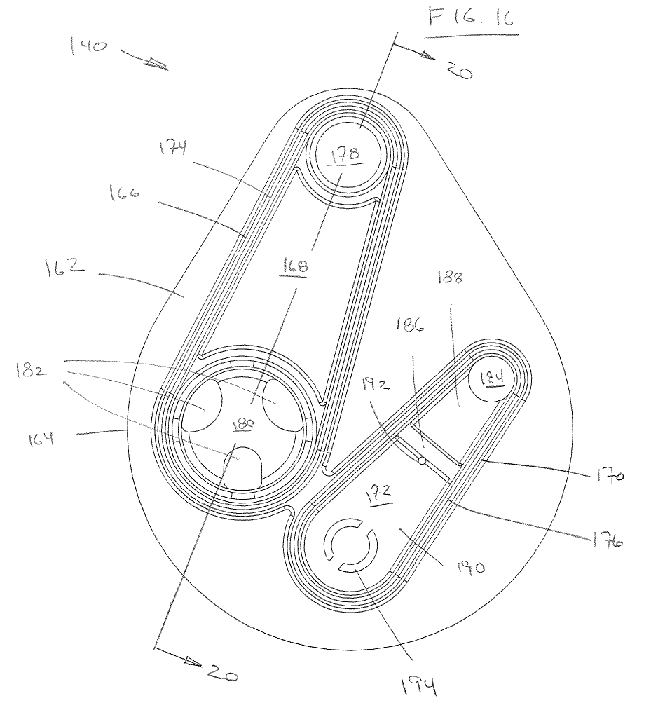

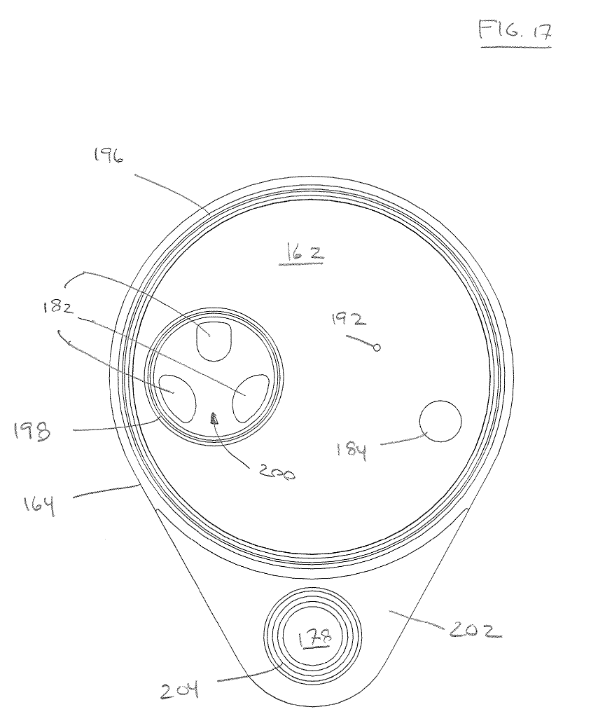

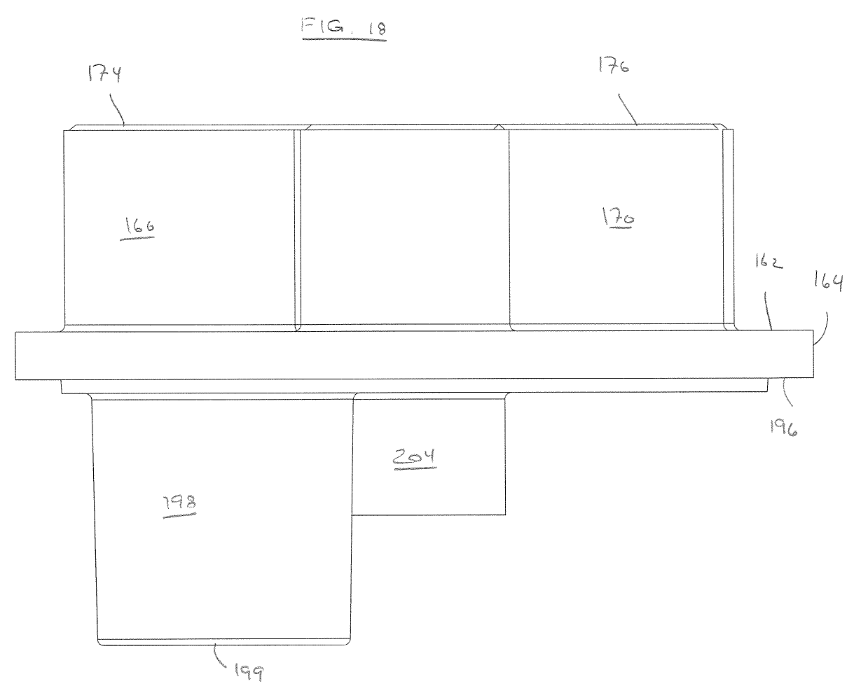

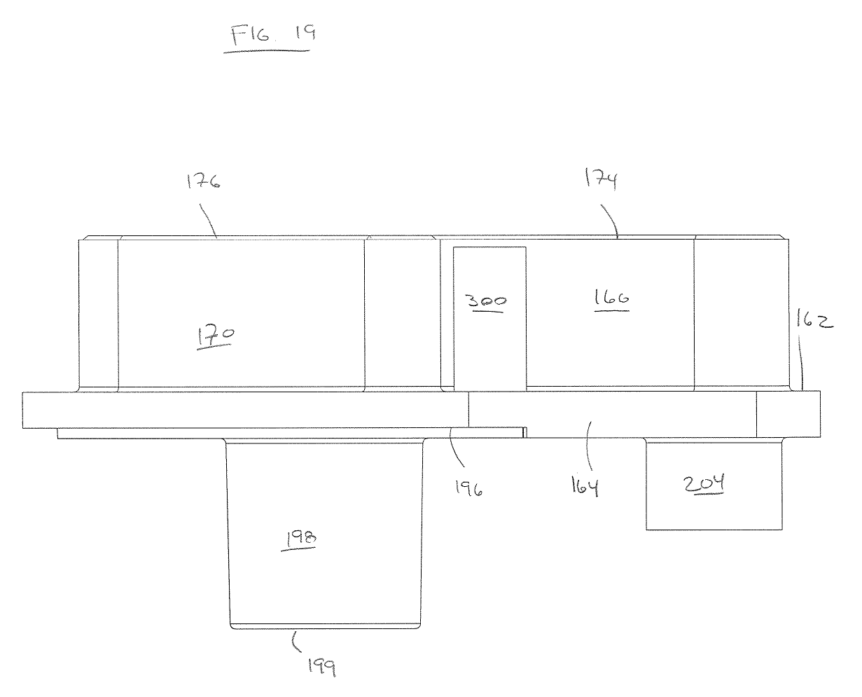

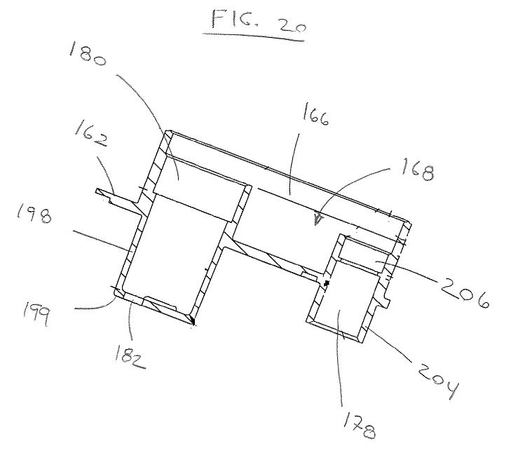

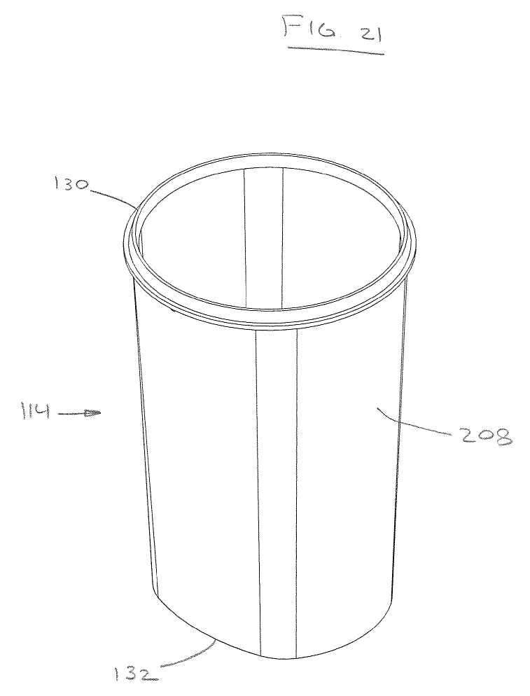

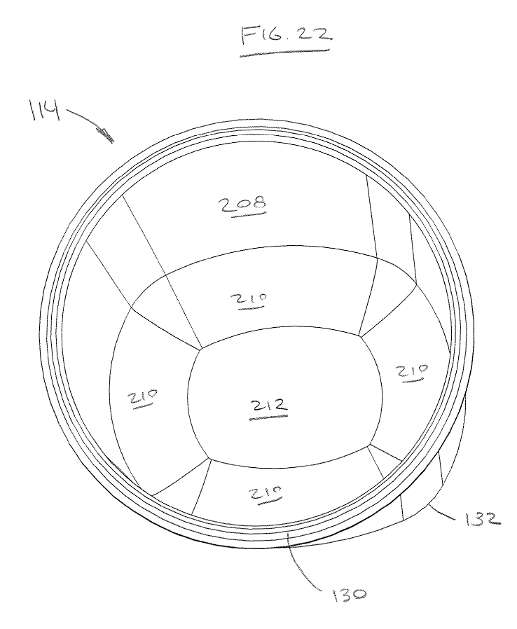

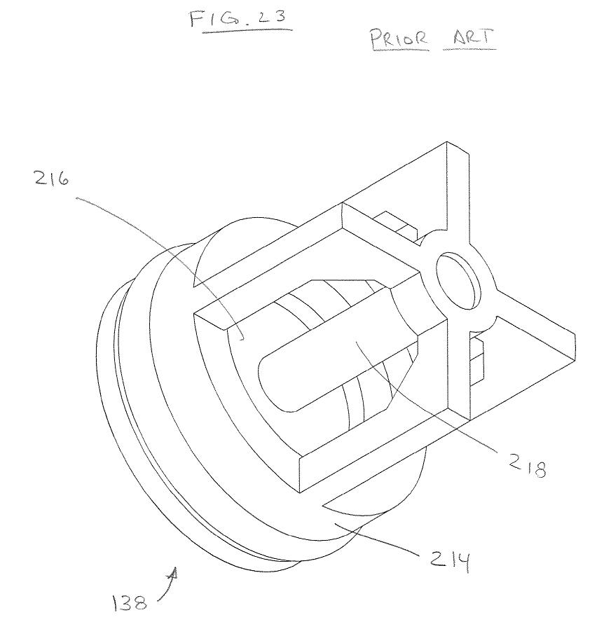

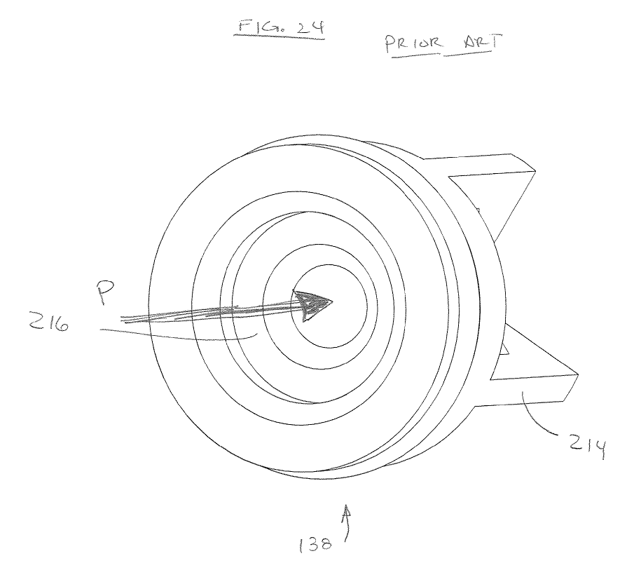

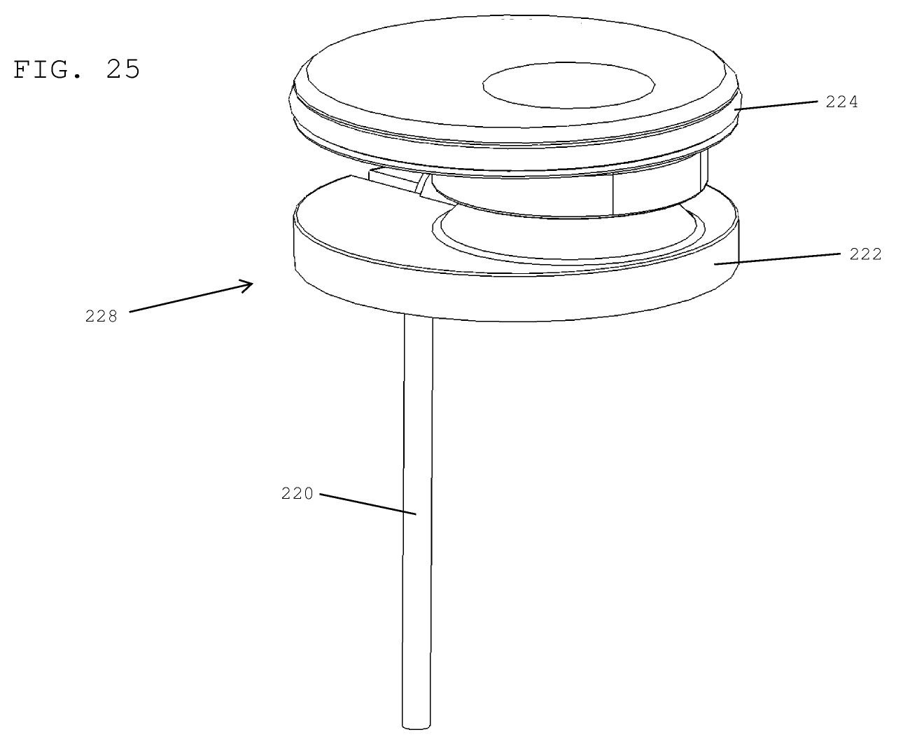

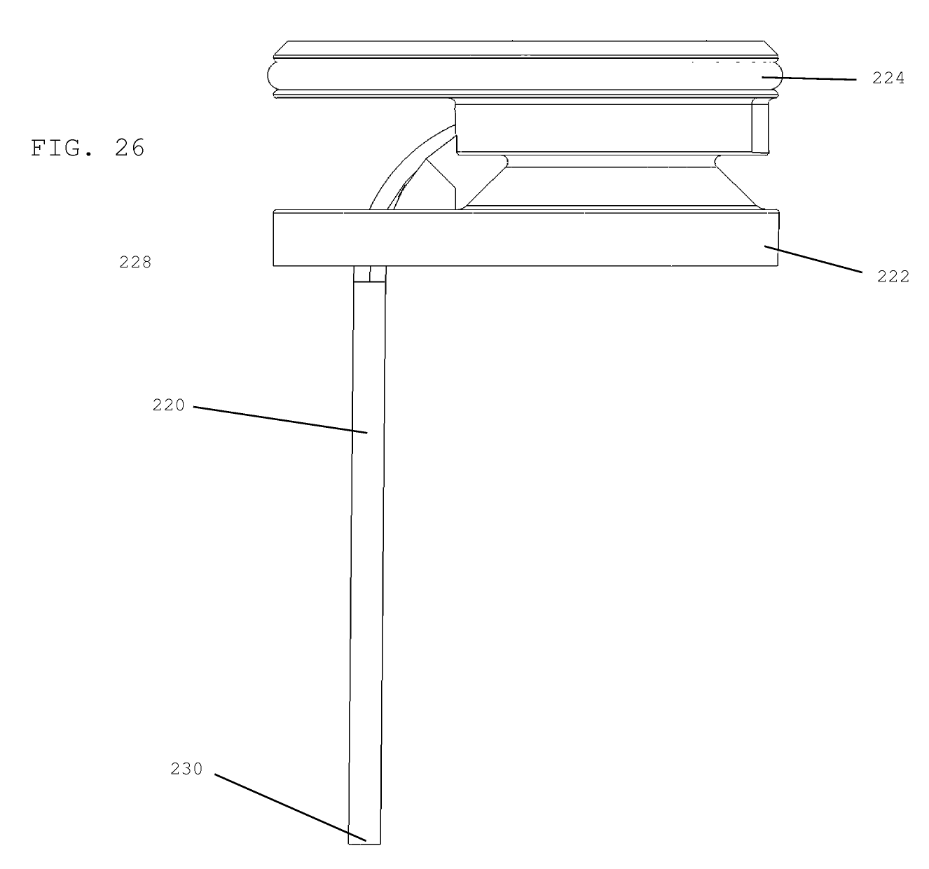

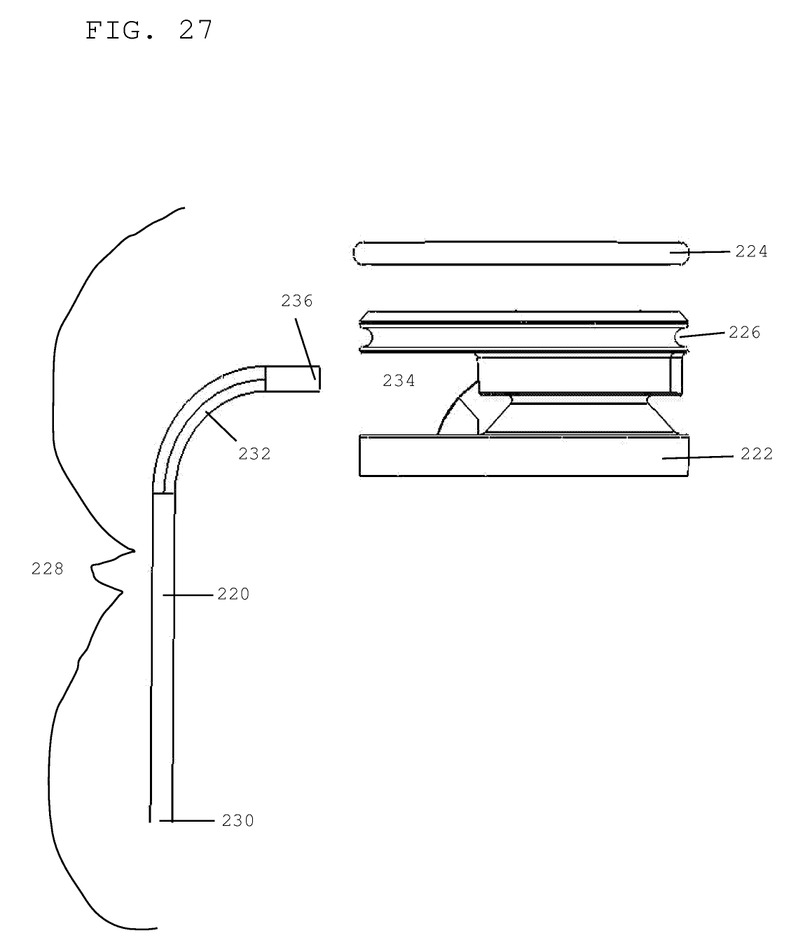

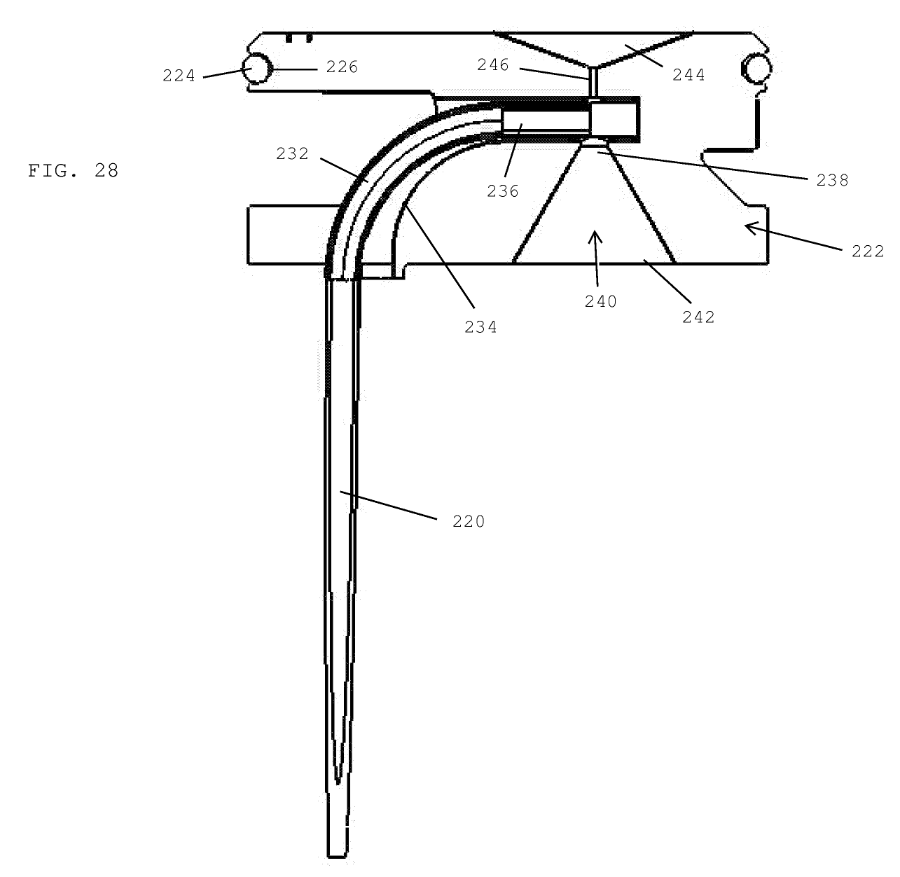

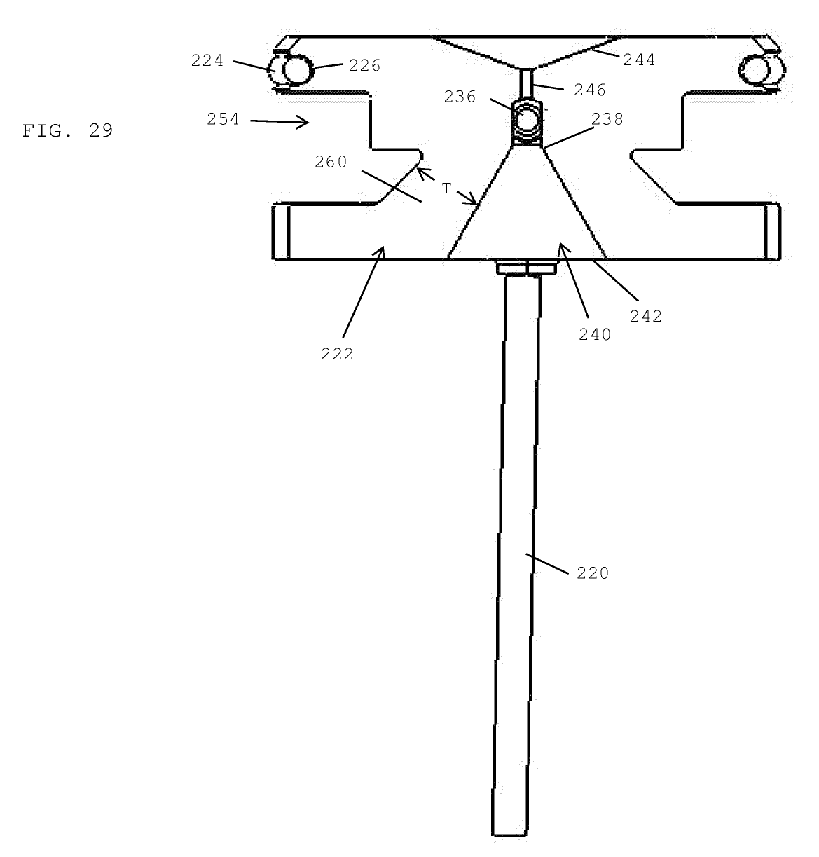

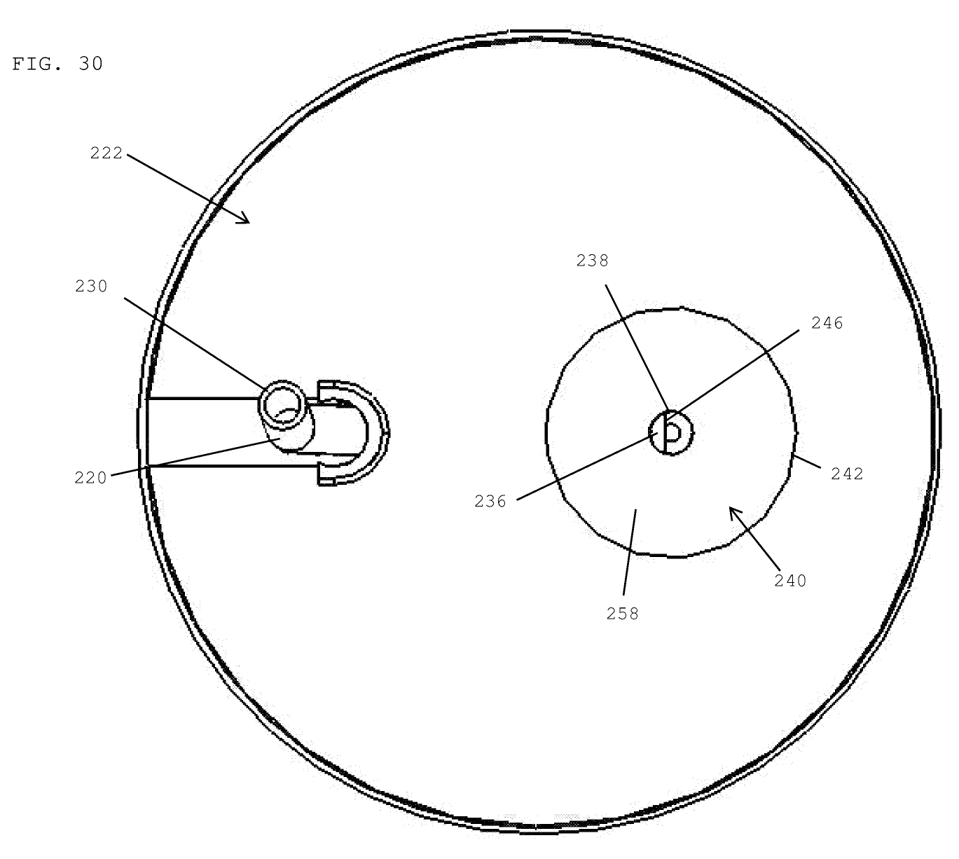

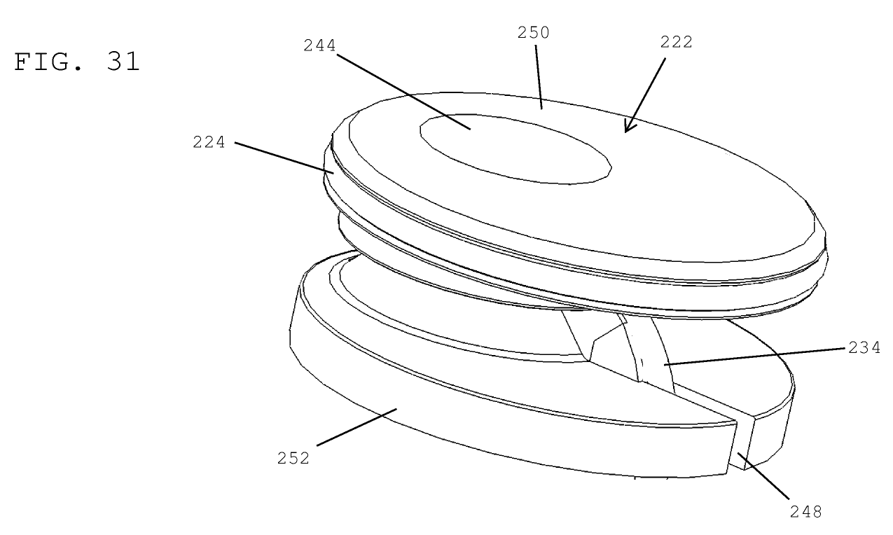

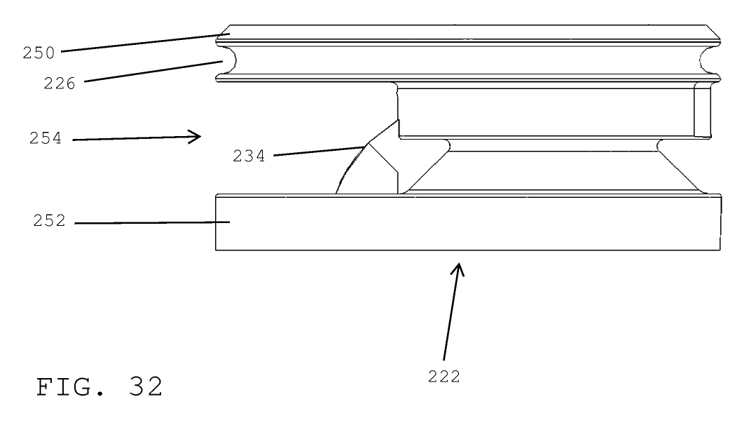

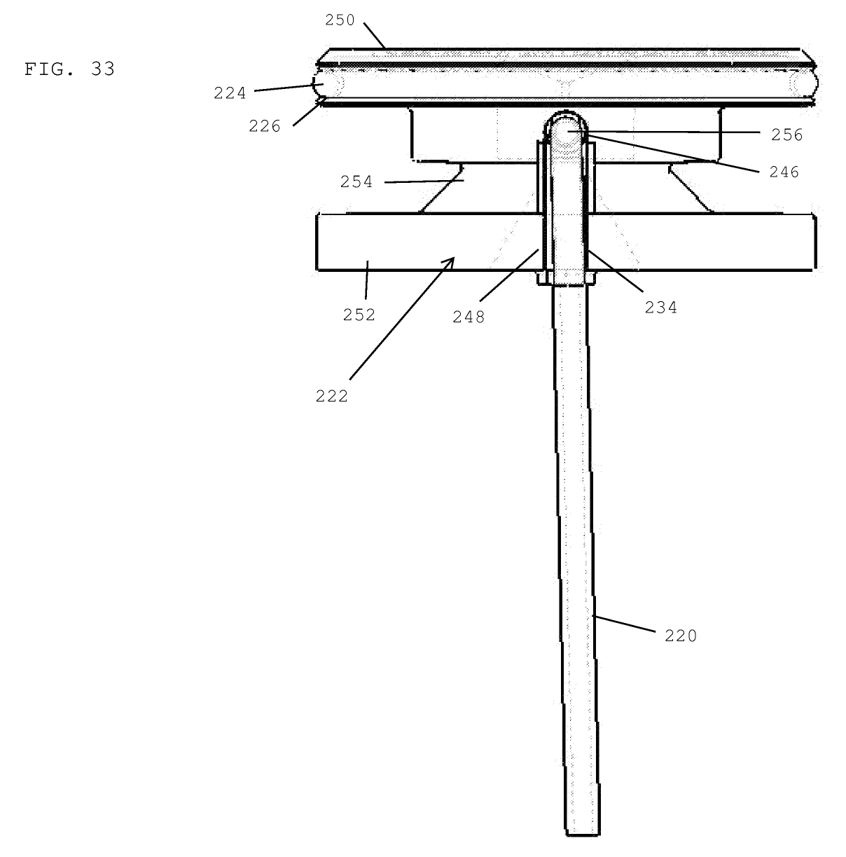

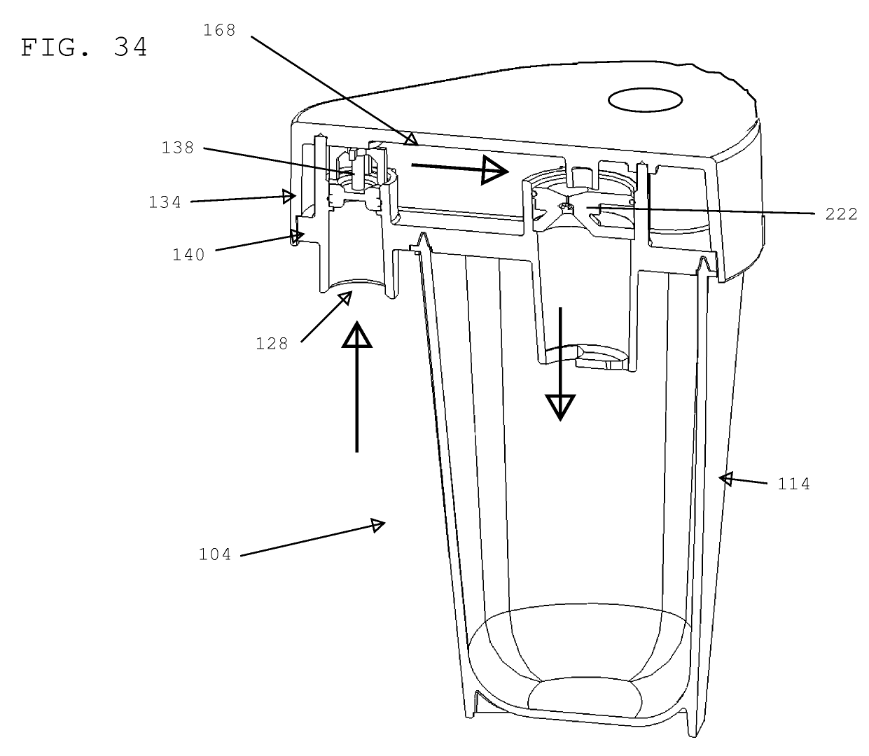

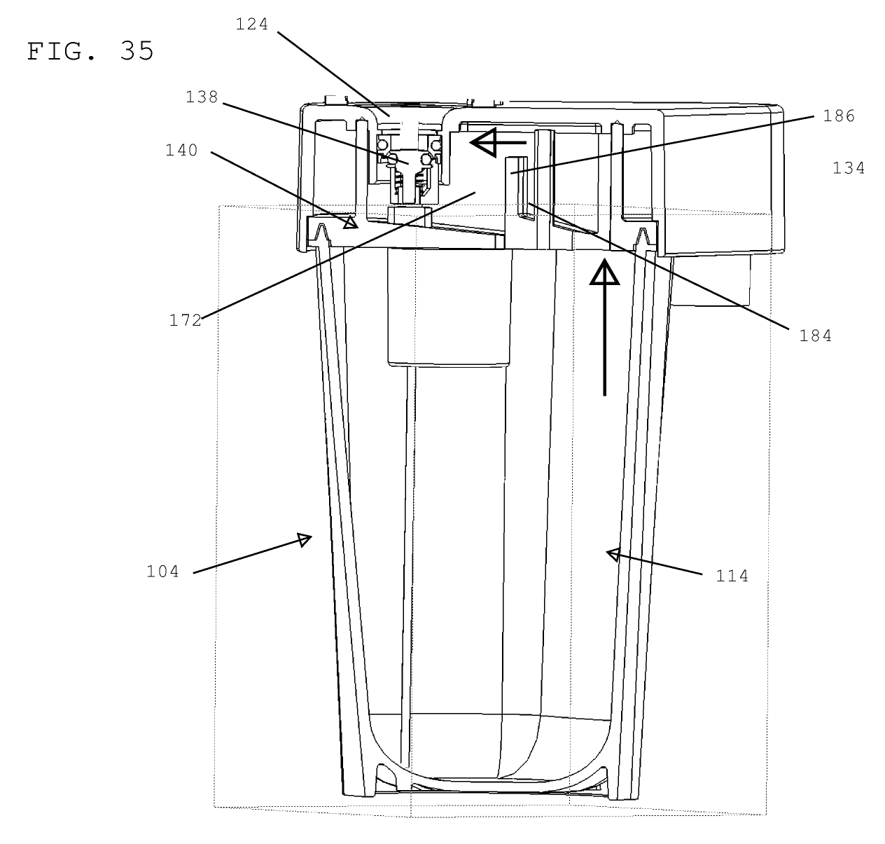

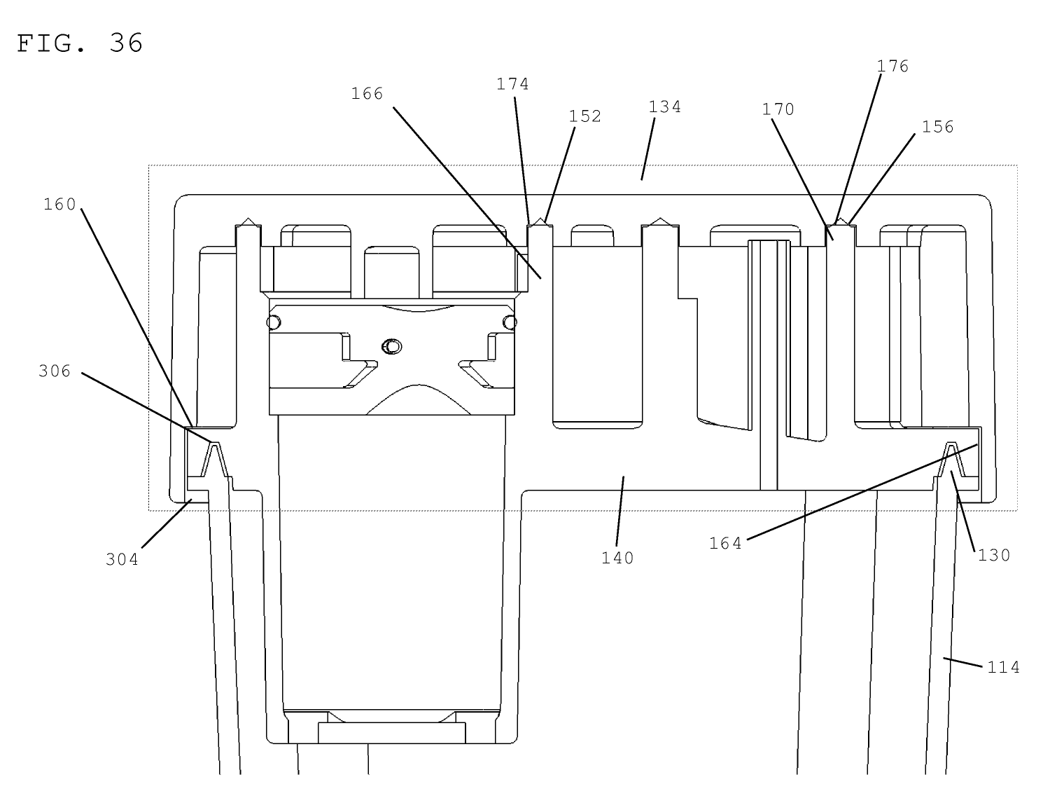

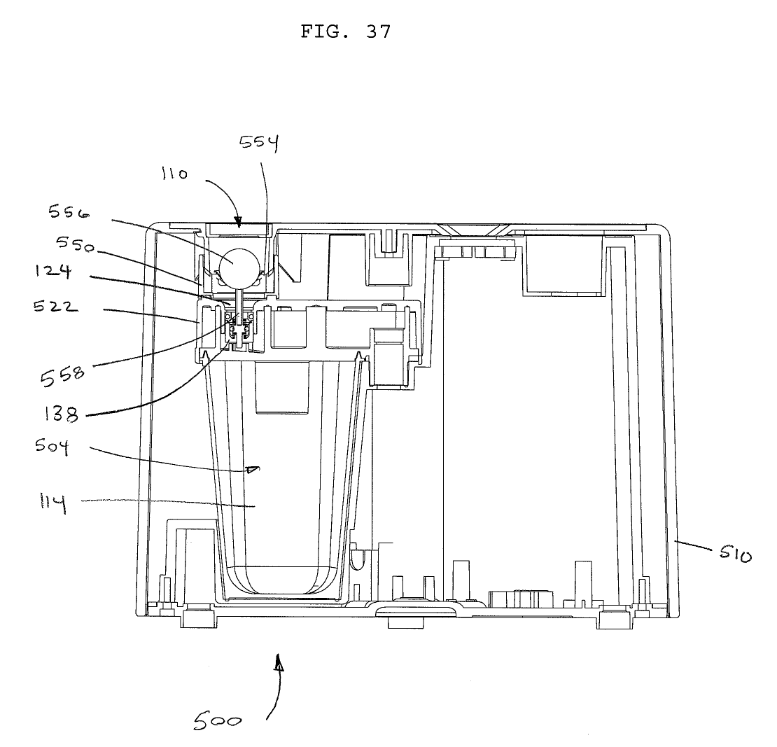

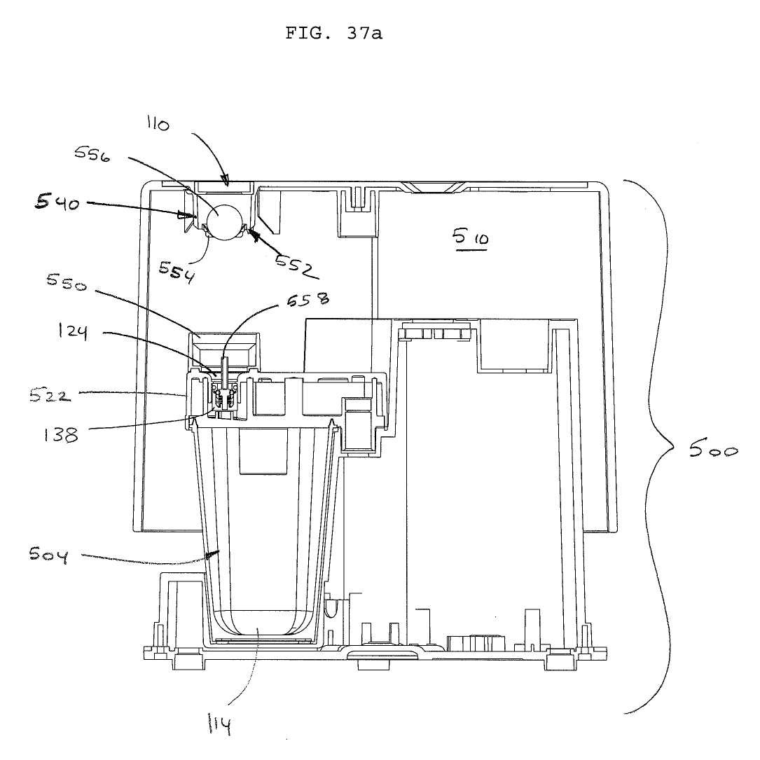

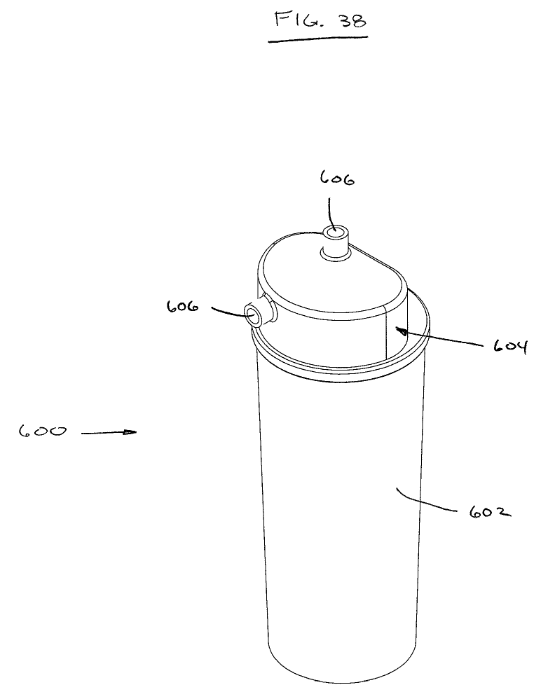

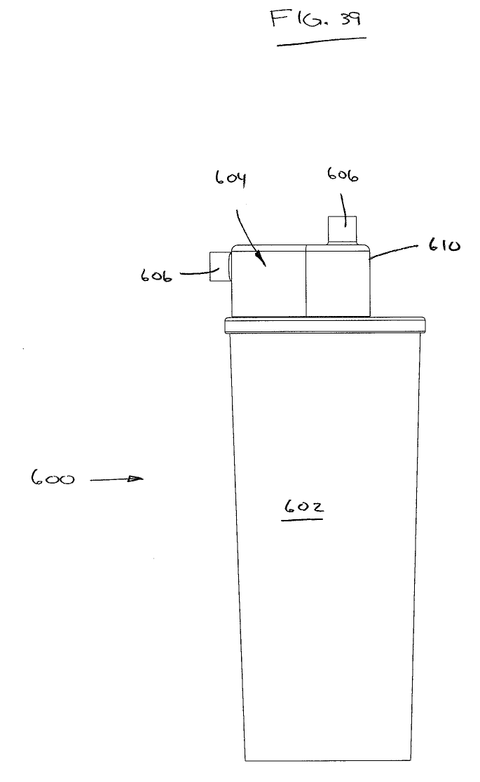

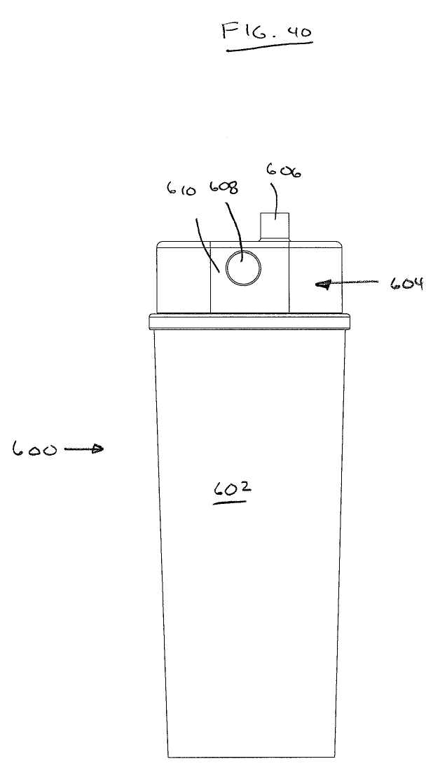

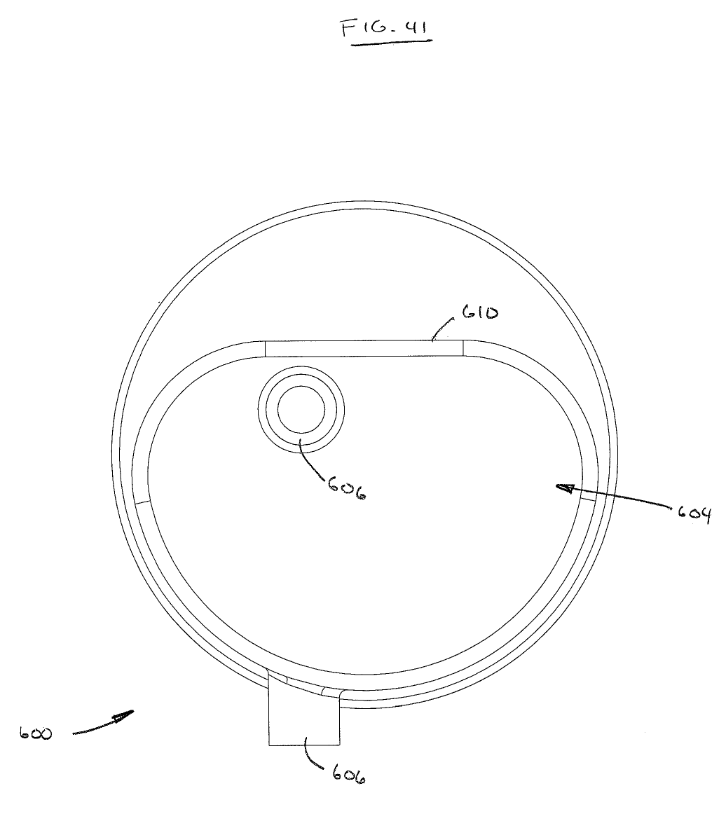

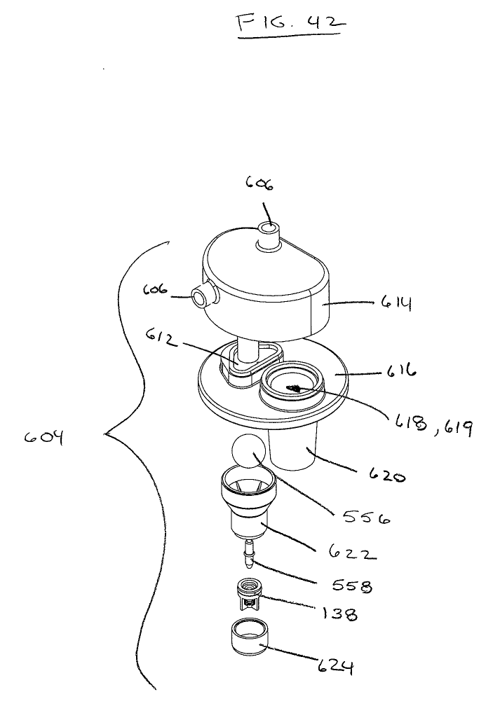

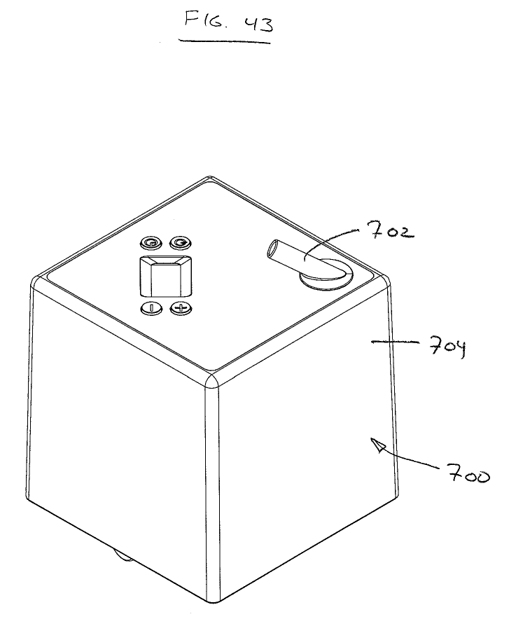

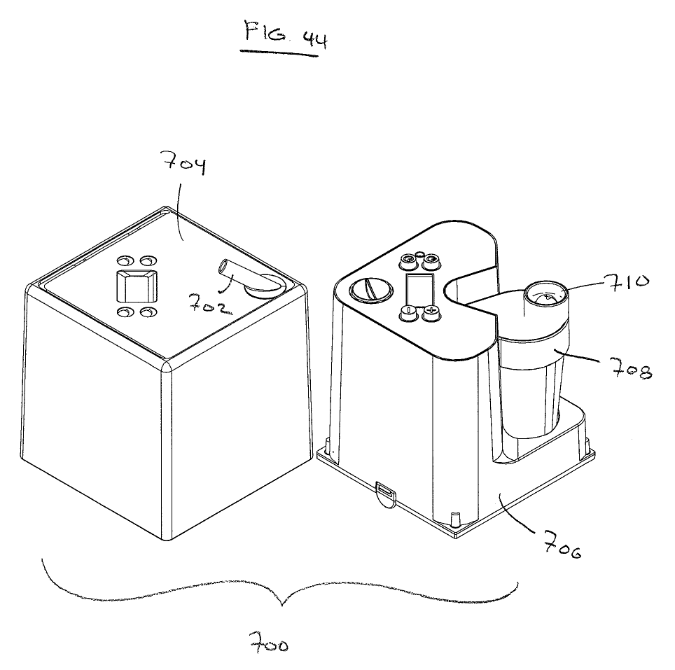

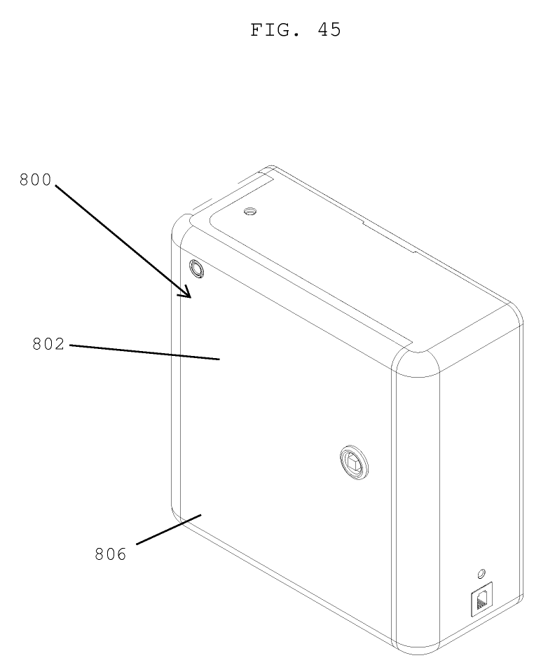

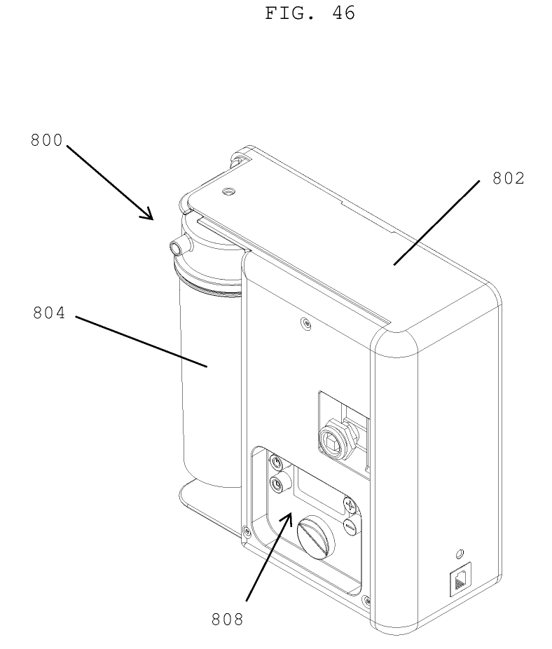

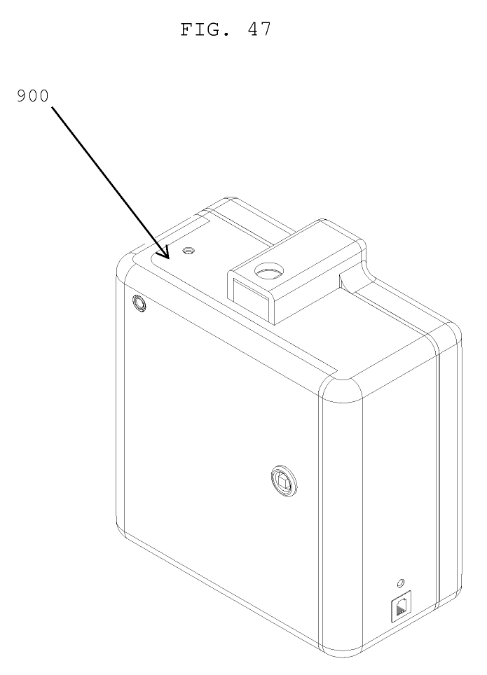

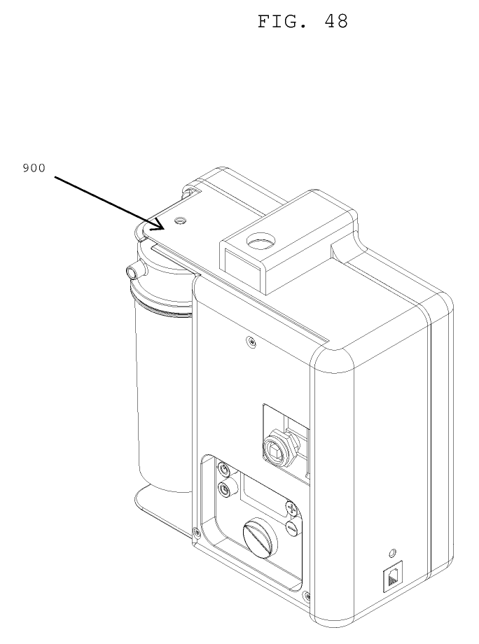

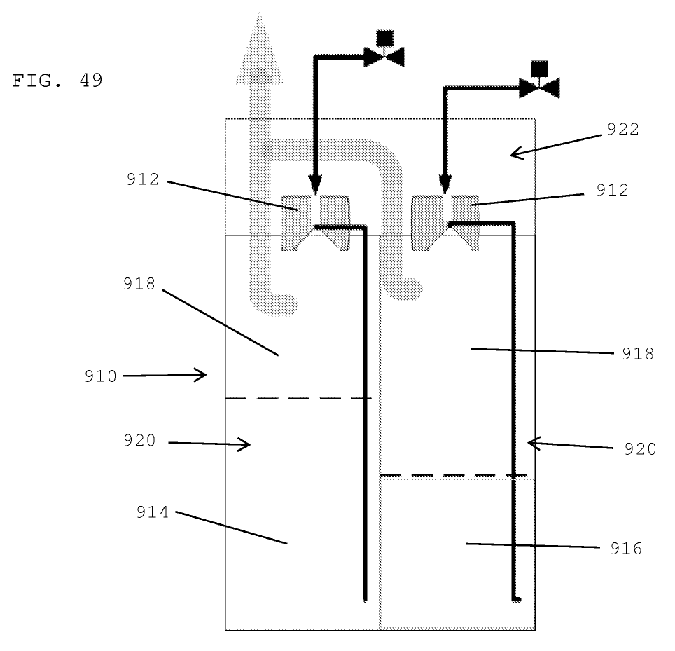

FIGS. 1 to 44 are taken from co-pending and co-owned U.S. patent application Ser. No. 11/734,660 (the '660 Application), the disclosure of which is incorporated herein by reference, and illustrate non-limiting examples of devices which may be configured to diffuse a glycol disinfecting compound according to the present disclosure. Co-pending and co-owned U.S. patent application Ser. No. 11/691,363 (the '363 Application), filed Mar. 26, 2007, the disclosure of which is incorporated herein by reference, provides disclosure of a system and method of controlling operation of a diffusion device such as may be found in the '660 Application.

It has been determined that the application or addition of excessive energy in a glycol disinfecting compound may adversely the affect the compound and possible compromise its effectiveness. So, a device that applies a minimum amount of energy to the compound to disperse the compound into the space to be treated is desirable. Device 10, as has been disclosed in the '660 Application, disperses or diffuses a liquid compound by use of a venturi and a source of pressurized gas, such as but not limited to an air compressor. Such an approach does not use heat or vibration on the liquid to be diffused and thus may be well suited to the dispersal of glycol compounds as described herein.

As shown in the '660 Application, a device for diffusing liquid such as a glycol disinfecting compound may include a cartridge with a reservoir including the compound and a diffusing head positioned on a top end. This diffusion head might include a venturi with a narrow end in fluid communication with a source of compressed air or gas and a siphon tube extending into the disinfecting compound. A large end of the venturi opens into an expansion chamber which is in turn open to an empty space above the liquid level within the reservoir. The empty space above the liquid level within the reservoir is in turn in fluid communication with an exit from the space into the diffusion head and then out of the cartridge.

In operation, pressurized air entering the venturi creates a low pressure zone within the venturi adjacent an upper end of the siphon tube. The low pressure draws the liquid compound from the reservoir into the venturi where it mixes with the air and becomes small droplets of compound mixed with the air in the expansion chamber. The expansion chamber includes side walls and an end baffle to aid in the collection and precipitation of larger droplets of the compound from the air. The larger droplets may be coalesce and drip back into the reservoir to be run through the venturi again. Once the air-compound droplet mixture exits the expansion chamber it enters the empty space above the liquid level within the reservoir. Dwell time of the mixture within this space permits more of the larger droplets to precipitate out and return to the reservoir liquid. As the mixture of air and compound works from the empty space within the reservoir through the exit to enter the space to be treated, additional structures may be included to encourage more large particles to be removed from the air/compound mixture.

When the air-compound mixture finally exits the cartridge, the droplets of compound within the mixture will be preferably sorted to include droplets in the range of about 200 nanometers to 10 microns. Most preferably, the size of the TEG droplets produced will be 500 nanometers or less in size. More common approaches to dispersion of droplets of glycol compounds may result in particle size distributions including much larger particle sizes. For example, a typical aerosol spray can might distribute particles which are all greater than 50 microns in size. As will be discussed below, it is desirable to diffuse smaller particles of the disinfecting compound as opposed to larger.

The principal purpose of treating the air within a space with TEG or a similar disinfecting glycol compound is to eliminate airborne contaminants. As such, any amount of the compound which falls out of the air and precipitates onto a surface within the space is not available to provide the desired effect. At the same time, it is desirable to have the greatest possible concentration of the compound in the air to provide the greatest level of disinfection of the air within the space. While it may be desirable to avoid precipitation of liquid compound on surfaces within the space, the gaseous compound in the air within the space will contact contaminants on these surfaces or within crevices, cracks or other surface imperfections that the liquid compound might not be able to treat.

The maximum amount of compound that may be present in the air, defined as the saturation point, may be determined from the atmospheric pressure and the humidity, among other conditions, within the space to be treated. Once this theoretical saturation point (defined as a concentration or mass of TEG per volume of space) is calculated, a device for diffusing TEG into the space may be activated to provide a measured dose of TEG into the space to achieve the desired concentration. When treatment of a space is initiated, a bolus of the compound may be released into the space to quickly raise the level of concentration to the desired level. Once this level has been reached, then the amount of compound to be released is preferably based on an expected or actual degree of spontaneous degradation or reduction of TEG within the space. This degradation might result from natural activity, such as the TEG interacting with contaminants in the air or from other causes.

In the discussion above, it should be noted that a diffusion device according to the present disclosure is actually releasing the TEG compound in a liquid form. To perform the disinfection function, TEG must be in a vaporous form. To go from the liquid released by the diffusion device to the disinfecting vapor, the TEG must evaporate from the droplets of liquid into the air. It is a well known physical phenomenon that a reduction of the surface area versus the mass of a liquid will to increase the tendency of the liquid to evaporate. The greater ratio of surface area to mass will result is an increase in the vapor pressure of the liquid.

### Creating Vapor From Liquid State TEG

Hanging the soaked and cured bed sheets served to massively increase the exposed surface area to volume ratio of the TEG liquid thus increasing reactivity because more TEG is exposed to react with the atmosphere. In effect, the vapor pressure or volatility of the TEG is increased by increasing the exposed surface area to volume ratio. The problem here was producing the quantity of TEG needed to sterilize a space without wasting material by having a higher than necessary concentration.

As noted above, TEG in its natural liquid state is very non-volatile. Making precise concentrations of TEG by the use of heat requires boiling a precise quantity of TEG that matches the sterilization concentration parameters of an enclosed space of known size and environmental condition. This was relatively easy to do in the experiments cited above because liquid-state TEG was placed into the test chamber in a precise quantity that produced exactly the concentration necessary for sterilization within the 15 to 20 minute window the researchers measured.

If there is a need for continuous sterilization or sterilization for discrete periods of time, simplified vapor production and the ability to control quantity and rate of injection into the space to be sterilized is essential. Without this achieving the right concentration would be by trial and error and then only for a moment. Scalability of the effort would be virtually impossible.

The cured and soaked bed sheets addressed the generation of vapor by a massive increase in the exposed surface area to weight ratio thus increasing volatility and vapor pressure. Control was nearly impossible. The bed sheets were hung in the test room. An optical refraction method was used to measure the quantity of TEG in the air. Once the desired concentration was reached, the bed sheets would be removed. Increasing the exposed surface produced TEG vapor molecules but the quantity could only be set once and not maintained for continuous sterilization at a desired level.

The present disclosure includes devices and methods to control the quantity of TEG vapor produced and injected into a space to reach the desired concentration level and maintain it at effective levels continuously. TEG vapor is produced not by boiling but by devices according the present disclosure which convert liquid-state TEG into droplets measuring less than 500 nanometers in diameter for injection in controllable quantity into the space to be sterilized.

Like the bed sheets of early researchers, transforming liquid TEG into nano-scale droplets affects a massive increase in the exposed surface area to weight ratio. The surface area of a droplet is calculated by the equation 4πr² where r is the radius of the droplet. The volume of a droplet is (4/3)πr³. A nano-scale droplet has a much greater surface area to weight ratio than the bed sheet. The total exposed surface area-to-volume ratio of a liquid is increased but not linearly as the surface area to weight ratio decreases.

A naturally occurring form of a droplet of liquid is a generally spherical shape. For a sphere, the ratio of internal volume is not directly proportional to surface area as the radius of the sphere changes. Smaller droplets have a much greater ration of surface area to mass and thus a higher vapor pressure. For droplets of the same compound subjected to the same atmospheric conditions, a smaller droplet will tend to evaporate faster than a larger droplet.

As mentioned above, other ways of encouraging evaporation of a compound are to raise its temperature or to otherwise impart some energy into the compound such as by subjecting the compound to ultrasonic vibrations. However, these approaches may harm the chemical or physical structure of the compound or may not create a desirable uniform droplet size to have consistent vapor pressure and thus evaporation.

Whether initiating treatment of a space or maintaining a level of treatment within a space, it is desirable to have the liquid compound be released into the air as a vapor within the space as rapidly as possible. A delay between the release of the compound and the rise of concentration of the vapor within the within space may result in lessened control of the concentration within the space or a failure of the concentration to be adapted to prevailing environmental conditions within the space. For example, a sudden influx of untreated air may be introduced within the space, resulting in a rapid drop in the concentration of TEG within the space. To raise the concentration of TEG within the newly altered air, a bolus of a liquid TEG compound may be injected into the air immediately upon the lowered concentration being detected. However, the concentration of the TEG vapor will not increase until the TEG within the liquid compound goes into vapor form. To accelerate the rise of concentration of vaporous TEG, it is desirable to release the bolus of liquid TEG compound in a form most suited to flash or evaporate.

Alternatively, if a super-saturation condition is detected, stopping or slowing the release of the liquid TEG compound into the space may be the response. However, the amount of liquid TEG compound remaining in suspension in the air that may become vaporous TEG will continue to increase the concentration of TEG above the desired or maximum efficient level. This would be wasteful of the TEG and may result in precipitation of the TEG from the air onto surfaces within the space.

Improved speed of evaporation of TEG released as liquid compound within a space to be treated will result in improved accuracy of control of the level of concentration of TEG vapor within the space. Improved accuracy of control may lead to improved efficiency in the treatment of such spaces with TEG. Improved methods of producing liquid TEG compound in a form accelerating evaporation without chemically or mechanically altering the TEG within the liquid compound may also serve to increase the efficiency of treatment of these spaces.

One conventional approach to improving the ability of moving TEG from a liquid form to a vaporous form has been to dissolve the TEG within a solvent having a much higher vapor pressure. Such an approach has several drawbacks, all of may be addressed by the approach described herein. First, such a solution will likely have a much higher amount of solvent as compared to TEG. Prior art references or products have disclosed solutions with as little as 4 to 6% TEG, with the remainder of the solution made up of solvents. Thus, a much high amount of liquid will need to be dispersed within the space to be treated to get the desired amount of TEG into the air. It is anticipated that a liquid TEG compound having as much as 86% or more TEG may be used with the device described above and the method and approach to TEG dispersion described herein.

Secondly, such approaches do not address the vapor pressure of TEG in liquid form, which as discussed above, is greatly impacted by the size of the droplet of TEG released. If the droplets of TEG left on the air after the solvent or carrier evaporates are too large, the same issue with the speed of conversion of TEG into vapor will apply. In addition, if droplets of TEG left in the air are too large, they may precipitate out of the air before having a chance to fully evaporate.

Thirdly, the solvent used as a carrier for the TEG within the solution will have to carefully chosen so as to be non-reactive with the TEG and also as innocuous as possible within the environment to be treated. Due to the ratio of solvent to solute in the conventional TEG liquid compounds, a great deal of the carrier will wind up in the atmosphere within the space to be treated, as compared to compounds that may be dispersed by the devices and methods described herein.

In addition to have a desirably small size droplet emitted by the devices and methods of the present disclosure, these devices and methods also provide a more uniformly sized droplet. This uniformity of size further enhances the accuracy of the initiation and maintenance of a desired concentration of TEG within a space. By having droplets more uniform in size, the rate of evaporation of liquid TEG from the droplets into the air within the space is more consistent. Consistent rates of evaporation permit better modeling of the impact of the release of a particular amount of TEG in liquid form on the concentration of vaporous TEG within the space.

Having greater control in the amount of TEG entering the air within a space, and a shorter time between release of liquid TEG and a concomitant rise in vaporous TEG concentration permits even more precise control of disinfection of a space when tied to a glycostat or other device directly measuring the concentration of TEG within the space. When a decrease from a desired level of concentration is detected, a diffusion device such as described above can be immediately activated to release a bolus precisely gaged to bring the concentration back to the desired level. When an increase above the desired level of detected, an immediate reduction or cessation in the operation of the diffusion device can be initiated to permit the level of TEG to reduce back to the desired level. Precision in timing of the reduction or cessation is desired to prevent overshooting the desired level and having to immediately release a bolus to then bring the level back up.

It may be desirable to operate the diffusion device to provide for a continually varying level of concentration with a space. Such a variation may be used to place sufficient liquid TEG into the air to provide for treatment at or above a desired concentration for some time and then cycling the diffusion device off. While the device is off, some of the vaporous TEG would naturally decay or interact with contaminants for be removed from the air. The liquid TEG could then vaporize and replace the lost TEG vapor. At some point, the degradation would overwhelm the amount of liquid TEG available for replenishment and at that time, the diffusion device could be cycled on again. Determination of when and for how long to operate the diffusion device to dispense liquid TEG may be made by modeling the space to be treated and using theoretical parameters to control operation, tying the operation to a glycostat or other device measuring vaporous TEG in the space, or a combination of such methods.

While the invention has been described with reference to preferred embodiments, it is to be understood that the invention is not intended to be limited to the specific embodiments set forth above. Thus, it is recognized that those skilled in the art will appreciate that certain substitutions, alterations, modifications, and omissions may be made without departing from the spirit or intent of the invention. Accordingly, the foregoing description is meant to be exemplary only, the invention is to be taken as including all reasonable equivalents to the subject matter of the invention, and should not limit the scope of the invention set forth in the following claims.

1. A method of treating an enclosed space with a liquid disinfecting agent, the method comprising:
providing a diffusion device with a source of compressed gas, a reservoir including the disinfection agent, and a venturi for spraying the agent into the droplets;
creating droplets of the disinfecting agent within the diffusion device, the droplets being less than about 10 microns in size;
dispersing the droplets from the diffusion device into the space to be treated so that the disinfecting agent in the droplets evaporates within the space.

2. The method of claim 1, further comprising the droplets being 500 nanometers and smaller in size.

3. The method of claim 1, wherein the disinfecting agent is triethylene glycol.

4. The method of claim 1, further comprising controlling the diffusion device to provide a concentration of the disinfecting agent within the space that is approximately the saturation level of the disinfecting agent.

5. The method of claim 4, further comprising monitoring the atmospheric conditions within the space and determining the saturation level from the atmospheric conditions.

6. The method of claim 4, further comprising operating the device to maintain the concentration of the disinfecting agent at approximately the saturation level of the disinfecting agent.

7. The method of claim 5, further comprising operating the device to maintain the concentration of the disinfecting agent at approximately the saturation level of the disinfecting agent and adjusting the operation of the machine as atmospheric conditions change to alter the saturation level in the space.

8. A device for diffusing a liquid disinfecting agent within a space to be treated, the device comprising:
a reservoir containing the liquid disinfecting agent and a head space within the reservoir above the level of the liquid;
a source of pressurized gas;
a venturi in fluid communication with the source of pressurized gas and with the reservoir;
wherein the flow of the pressurized gas into the venturi creates a vacuum to draw liquid disinfecting agent into the venturi where the liquid and gas mix;
the venturi further comprising an outlet through which the mixed gas and liquid pass into the head space of the reservoir;
the reservoir further comprising an outlet to release the mixed gas and liquid from the head space into the space to be treated;
wherein the mixed gas and liquid entering the space to be treated from the outlet includes droplets of the liquid disinfecting compound less than about 10 microns in size; and,
wherein the disinfecting compound evaporates from the liquid droplets into a gaseous form within the space to be treated, providing a concentration of disinfecting compound with the space.

9. The device of claim 8, further comprising a programmable controller to control operation of the device and to turn the device on, permitting pressurized gas to flow into the venturi, and off, stopping flow of pressurized gas into the venturi, to vary the concentration of the gaseous disinfecting compound within the space to be treated.

10. The device of claim 9, wherein the operation of the device is varied based on the atmospheric conditions with the space.

11. The device of claim 10, wherein the operation of the device is varied to maintain a concentration of the gaseous disinfecting agent within the space approximately at the saturation point.

12. The device of claim 11, further comprising an atmospheric condition sensor within the space to be treated, the sensor coupled to the controller, wherein the sensor provides input to the controller to vary operation of the device to maintain a concentration of the gaseous disinfecting agent with the space based on the atmospheric conditions sensed.

13. The device of claim 11, further comprising a concentration sensor within the space to be treated, the concentration sensor coupled to the controller, wherein the sensor provides input to the controller of the concentration of the gaseous disinfecting agent within the space to be treated and the controller varies operation of the device based on the relationship of the sensed level to the saturation level of the gaseous disinfecting agent within the space to be treated.

14. The device of claim 8, wherein the mixed gas and liquid entering the space to be treated from the outlet includes droplets of the liquid disinfecting compound less than about 500 nanometers in size.

15. The device of claim 8, further comprising the reservoir outlet in fluid communication with the space to be treated through an air distribution system.

16. A method of disinfecting a space, the method comprising:
placing a diffusion device in communication with the space;
dispensing droplets of a liquid disinfecting agent from the diffusion device into the space;
monitoring the concentration of vapor of the disinfecting agent within the space; and,
adjusting the dispersion of droplets into the space based on the measured concentration of vapor.

17. The method of claim 16, the disinfecting agent further comprising triethylene glycol.

18. The method of claim 16, further comprising monitoring the atmospheric conditions within the space and adjusting the dispersion of droplets into the space to maintain the concentration of vapor near a saturation point within the space.

19. A method of treating an enclosed space with a liquid disinfecting agent, the method comprising:
providing a diffusion device with a source of compressed gas, a reservoir including the disinfection agent, and a venturi for spraying the agent into the droplets;
providing the disinfection agent includes at least fifty percent triethylene glycol;
dispersing the droplets from the diffusion device into the space to be treated so that the disinfecting agent in the droplets evaporates within the space.

20. The method of claim 19, wherein the disinfection agent includes at least eighty-six percent triethylene glycol.
